(19) United States
(12) Patent Application Publication
(10) Pub. No.: US 2017/0216519 A1
(43) Pub. Date: Aug. 3, 2017

(54) WEARABLE FLUID DISPENSING DEVICES, SYSTEMS AND METHODS RELATED THERETO

(71) Applicant: Preciflex SA, Neuchâtel (CH)

(72) Inventors: Lucien VOUILLAMOZ, Feusisberg (CH); Gregory DOURDE, Neuchâtel (CH); Michel ORSINGER, Bursinel (CH)

(73) Assignee: Preciflex SA, Neuchâtel (CH)

(21) Appl. No.: 15/515,206

(22) PCT Filed: Oct. 5, 2015
(86) PCT No.: PCT/IB2015/001777

§ 371 Date: Mar. 29, 2017

Related U.S. Application Data
(60) Provisional application No. 62/059,755, filed on Oct. 3, 2014.
(60) Provisional application No. 62/059,732, filed on Oct. 3, 2014.

(51) Int. Cl. A61M 5/142; A44C 15/00; G04G 21/04; G04B 37/12; G04B 47/06; G04G 21/02; A61M 5/172; A44C 5/00

(52) U.S. Cl. CPC A61M 5/14244; A61M 5/1723; A44C 15/002; A44C 15/003; A44C 5/0023; A44C 5/003; G04B 37/127; G04B 47/063; G04G 21/02; G04G 21/04; A61M 2005/14272; A61M 2005/1726; A61M 2205/3372; A61M 2205/3584; A61M 2205/3592; A61M 2205/3606; A61M 2205/6009; A61M 2005/14268; A61M 2205/18; A61M 2205/581; A61M 2205/583; A61M 2205/502; A61M 2205/3303; A61M 2205/276; A61M 2205/8206; A61M 2209/045

(57) ABSTRACT

A system is provided for dispensing a fluid. The system has a housing having a fixation means to a user and an orientation element; the cartridge formed so as to be held in a given orientation by the orientation element with respect to the housing; and control means to activate the cartridge to eject a fluid. In one aspect, the system is a emergency rescue fluid(s) transdermal delivery system which includes a removable, single use emergency rescue fluid(s) dispensing cartridge, a wearable device into which the emergency rescue fluid(s) dispensing cartridge uniquely, matingly and removably inserts. The system includes a communication pathway between the wearable device and at least one other cloud network node, and/or at least one communication pathway between the wearable device and at least one GPS network node. In another aspect, the system dispenses a perfume or other fragrance.

### CROSS REFERENCE TO RELATED APPLICATIONS

This PCX application claims priority to U.S. Provisional Application No. 62/059,755, and 62/059,732, both filed Oct. 3, 2014, the contents of which are incorporated by reference hereto.

### COPYRIGHT & LEGAL NOTICE

A portion of the disclosure of this patent document contains material which is subject to copyright protection. The Applicant has no objection to the facsimile reproduction by anyone of the patent document or the patent disclosure as it appears in the Patent and Trademark Office patent file or records, but otherwise reserves all copyright rights whatsoever. Further, no references to third party patents or articles made herein is to be construed as an admission that the present invention is not entitled to antedate such material by virtue of prior invention.

### BACKGROUND OF THE INVENTION

This invention relates to a fluid dispensing device to be worn by a user.

In one variant, the invention relates to an emergency rescue fluid dispensing connected wearable devices, systems and methods related thereto, and in particular emergency rescue fluid(s) dispensing wearable devices with an autonom time base, emergency rescue fluid(s) dispensing systems and methods.

There exists a need for a wristwatch or other wearable device such as a bracelet that serves several functions and provides for readily accessible emergency rescue fluid(s) transdermal injection by a man, woman or child, without the need for searching for a emergency rescue fluid(s) bottle in a purse, or having to carry a emergency rescue fluid(s) bottle with a user.

In another variant, the invention relates to fragrance dispensing connected wearable devices with an autonom time base, systems and methods related thereto, and in particular fragrance dispensing wearable devices with an autonom time base fragrance dispensing systems and methods.

There exists a need for a wearable device that serves several functions and provides for readily accessible fragrance dispersal by a man, woman or child, without the need for searching for a fragrance bottle in a purse, or having to carry a fragrance bottle with a user.

### SUMMARY OF THE INVENTION

An object of the invention is it to provide a system for dispensing a fluid, the system device to be worn by a user.

In one variant, such an object is attained with a system for emergency rescue fluid(s) transdermal delivery thereof. The system provides a previously filled, removable, single use, disposable emergency rescue fluid(s) cartridge, a wristwatch or other worn item (hereinafter referred to simply as a wearable device) into which the emergency rescue fluid(s) dispenser matingly and removably inserts

It is a further object of the invention to provide a method of operating at least a portion of a emergency rescue fluid(s) dispensing timepiece system. The method includes the steps of: providing a wearable device with a sealed fluid tight chamber, and providing a previously filled emergency rescue fluid(s) dispensing cartridge which matingly and removably fits into the sealed fluid tight chamber of the wearable device.

It is a further object of the invention to provide a system for fragrance atomization, vaporization and or general dispersion thereof. The system provides a removable, refillable or disposable fragrance cartridge, a wearable device into which the fragrance dispenser matingly and removably inserts, and a filler assembly for dispensing into the fragrance dispenser a fragrance. By fragrance, the term is meant to be interpreted in it's broadest sense, to include mosquito sprays, odours

It is a further object of the invention to provide a method of operating at least a portion of a fragrance dispensing timepiece system. The method includes the steps of: providing a wearable device with a sealed fluid tight chamber, and providing a fragrance dispensing cartridge which matingly and removably fits into the sealed fluid tight chamber of the wearable device wherein the cartridge operates (diffuses, vaporizes, atomizes the fragrance) completely separately from the ordinary time-keeping functions of the wearable device. The method includes one or more of these steps, alone or in combination, providing a filler of the cartridge for filling the cartridge with a fluid, manually filling the cartridge with fragrance via a filler by manually pressing the cartridge into the filler, pushing a button on the cartridge when installed in the filler such that the manual force on the button fills the cartridge with the fluid from the filler (manually filling further comprising inserting a needle into a septum of the cartridge thereby filling a reservoir of the cartridge), removing the cartridge from the filler, inserting the cartridge into a housing of the wearable device, and pushing a button on the cartridge such that the manual force on the button diffuses the fluid front the cartridge to the exterior of a user.

The object is attained with another variants of these inventions, embodied in a wearable device and/or wearable device/cartridge combination which are communicatively linked to each other, and communicatively linked to a network, including a cloud computer network, GPS satellite network, a mechanical or electronical time base and/or external electrical energy source.

### BRIEF DESCRIPTION OF THE DRAWINGS

FIG. 1 is a perspective view of a system of the present invention including a cartridge, wearable device, cloud, and external power source cable of the present invention.

FIG. 2 is a perspective view of a cartridge and cartridge filler of the present invention.

FIG. 3 is a perspective view of the assembly of the present invention with the cartridge removed.

FIG. 4 is a flow chart of a method of one variant of the invention.

FIG. 5 is a flow chart of a method of the second variant of the invention.

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended for distinguishing between similar elements and not necessarily for describing a sequential or chronological order. Moreover, relative terms like ‘front’, ‘back’, ‘top’ and ‘bottom’, and the like in the Description and/or in the claims are not necessarily used for describing exclusive relative position. Those skilled in the art will therefore understand that such terms may be interchangeable with other terms, and that the embodiments described herein are capable of operating in other orientations than those explicitly illustrated or otherwise described.

### DETAILED DESCRIPTION OF THE PREFERRED EMBODIMENT

The following description is not intended to limit the scope of the invention in any way as they are exemplary in nature, serving to describe the best mode of the invention known the inventors as of the filing date hereof. Consequently, changes may be made in the arrangement and/or function of any of the elements described in the exemplary embodiments disclosed herein without departing from the spirit and scope of the invention.

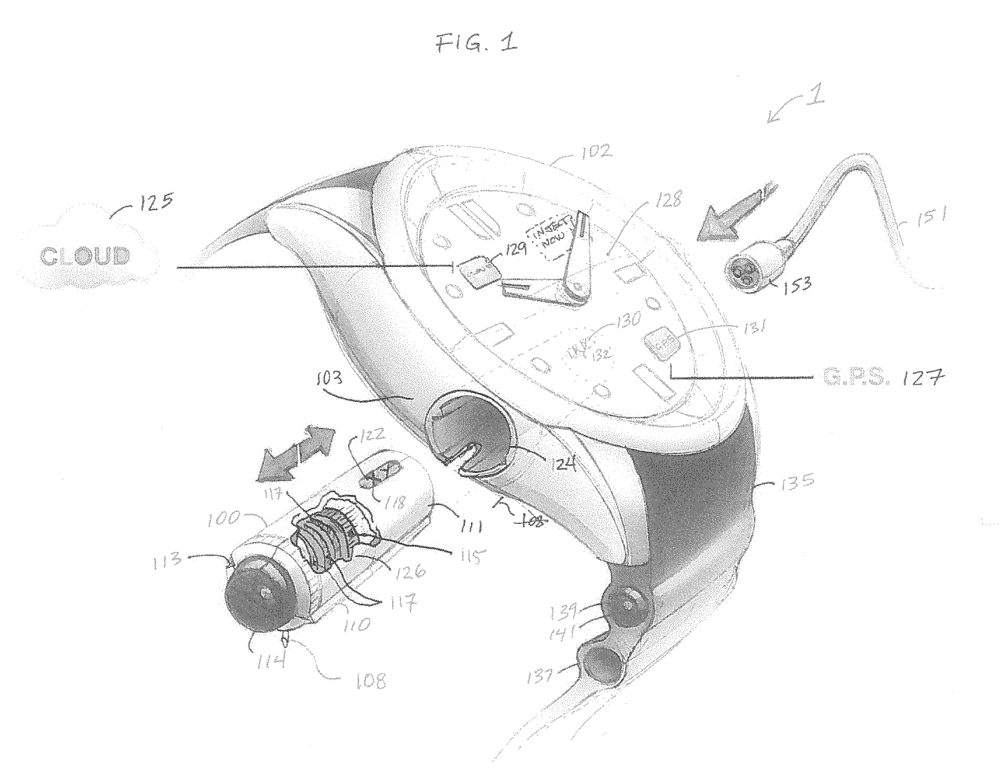

Referring now to FIG. 1, systems for emergency rescue fluid(s) transdermal delivery (or optionally, in another variant, atomization, vaporization and or generally dermal dispersion) are provided. System 1 provides a removable, fixed dose, micro-injector emergency rescue fluid(s) cartridge 100, a wearable device 102 into which the emergency rescue fluid(s) dispenser 100 matingly and removably inserts in a lock and key manner specific to the type of fluid 122 within the cartridge 100 (e.g. a medicament, an emergency anti-venom, an emergency drug or therapy to treat anaphalactic shock, bee stings, allergies, etc.).

The cartridge 100 is adapted in form from any one of the injectors described in PCT/US2012/048044, filed 25 Jul. 2012, the content of which is incorporated by reference hereto and relied upon.

The cartridge 100 and/or watch housing also includes one or more micro-needle(s) 108 that, after an injection for transdermal delivery of fluid 122 has been triggered, are retractable into the cartridge 100 (in a further variant having a disposable cartridge) or into the housing of the watch 102. Note that although the micro-needle 108 is shown extended, the cartridge 100, upon removal, would cause the needle to automatically retract into a recess to prevent injury to the user on ejection or insertion. After use, the cartridge 100 may be automatically ejected by a suitable mechanism. The cartridge 100 has integrated therein a blocking device (not shown) which blocks inadvertent ejection of fluid or injection of the fluid unless the cartridge is safely in the housing in a proper orientation. When used herein, “ejection” means exiting a fluid reservoir in the cartridge 100 and is meant to include the ejection of a fragrance as described below as well as the initiation of the process of injection into the user (namely, ejection of the fluid from the cartridge and then injection into the user). In other variants, a vaporizer or other assembly is used to release an emergency rescue fluid(s) into the atmosphere around a user. Cartridge 100 includes a protrusion 110 which extends outside of the housing 111 of cartridge 100 and one or more grooves 113. The protrusion 110 further includes an actuator 114. The actuator 114 is an emergency rescue fluid(s) dispensing manual actuator, a manually actuated pump, or in another variant. The actuator 114 and or the cartridge 100 further includes an optional ornamental crown. The ornamental crown is selected from materials including non-precious metals, precious metals, natural gemstones, synthetic gem stones, and/or diamonds for example. The cartridge 100 further includes a transparent viewing window 118 and includes an interior chamber. The interior chamber further includes a fluid 122. The fluid 122 is selected from the group consisting of an emergency rescue fluid(s), a treatment for a medical condition, a drug, a vitamin, a natural supplement, and the like. The fluid is preferably selected among possible fluids such as peptides, proteins, hormones including insulin, calcitonin, calcitonin gene regulating protein, atrial natriuretic protein, colony stimulating factor, betaseron, erythropoietin (EPO), interferons such as .alpha., .beta, or .gamma, interferon, somatropin, somatotropin, somastostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues or antagonists thereof, such as IL-1ra; analgesics such as fentanyl, sufentanil, butorphanol, buprenorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; anti-emetic agents such as scopolamine, ondansetron, domperidone, metoclopramide, and analogues thereof; cardiovascular agents, anti-hypertensive agents and vasodilators such as diltiazem, clonidine, nifedipine, veraparnil, isosorbide-5-monotritate, organic nitrates, agents used in treatment of heart disorders, and analogues thereof; sedatives such as benzodiazepines, phenothiazines, and analogues thereof; chelating agents such as defroxanune, and analogues thereof; anti-diuretic agents such as desmopressin, vasopressin, and analogues thereof; anti-anginal agents such as fluoronracil, bleomycin, and analogues thereof; anti-neoplastics such as fluorouracil, bleomycin, and analogues thereof; prostaglandins and analogues thereof; and chemotherapy agents such as vincristine, and analogues thereof, treatments for attention deficit disorder, methylphenidate, fluvoxamine, bisoprolol, tacrolimus, sacrolimus and cyclosporin, vitamins suspended in a liquid carrier, antivenoms, syrums, medications, antibodies, Actemra (tocilizumab), Adcretris (brentuximab vedotin), Arzerra (ofatumamab), Avastin (bevacizumab), Benlysta (belimumab), Cimzia (certolizumab pegol), Erbitux (cetuximab), Herceptin (trastuzumab), Humira (adalimumab), Ilaris (canakinumab), Lucentis (ranibizumab), Mylotarg (gemtuzumab ozogamicin), Perjeta (pertozumab), Prolia (denosumab), Remicade (infliximab), Simponi (golimumab), Soliris (eculizumab), Stelara (ustekinumab), Tysabri (natalzumab), Vectibix (panitumumab), Xgeva (denosumab), Xolair (ornalizumab), Yervoy (ipilimumab), and Zevalin (ibritumomab tiuxetan).

The cartridge 100 is configured so that functionality of the cartridge 100 for transdermal delivery of the fluid 122 is completely independent of the functioning of the wearable device 102. In another variant, the functionality of the wearable device is coupled with the dispensing data from the cartridge. By way of example, once cartridge 100 fluid 122 has been delivered to a user, the wearable device has electronic circuitry, algorithms, and communication circuitry to connect to a network such as the Cloud to deliver drug delivery data and/or user health data to another computer node communicatively linked to the wearable device. This data is correlated with GPS location data of the user wearing the watch 102 via a GPS network 127 and screens 129, 131 are provided for the user showing the status of various functions of the watch. Further, watch band 135 is provided with one or more compartments 137, 139 into which a spent, single use cartridge 141 is inserted or a reserve cartridge is stored until needed.

The wearable device 102 further includes a housing 103 having a chamber 124 which matingly and removably couples with an exterior housing 126 of cartridge 100 in a specific “lock and key” manner. The chamber 124 further includes a fluid tight chamber assembly within an interior housing portion of the wearable device 102. The exterior housing 126 of cartridge 100 is sealed and fluid tight, except for the drug delivery subsystem of the wearable device 102 and/or cartridge combination, e.g. at the drug delivery exit points. The wearable device 102 further includes a watch dial surface 128 further comprising a transparent portion 130, e.g. also including wording such as full or empty. The transparent portion 130 further includes a plurality of markings 132 indicating the amount of fluid 122 remaining in cartridge 100.

The cartridge 100 further includes a manual push actuator 114 for actuating transdermal delivery of fluid 122. In yet a further variant, the cartridge 100 further includes a piston 115 and spring 117 for pressurizing fluid within the internal chamber of the cartridge 100.

The combination of wearable device circuitry and communicative interaction with the cloud network permits the wearable device 102/cartridge 100 combination to function as a network node on the cloud network and transmit data to other network nodes on the cloud network or other networks, such as data related to whether a drug delivery has been made, data related to the location of a user who has received a drug delivery, the location of a soldier on a battle field which has received, e.g. a drug or other anti-dote, etc. The system 1 also includes an external power cable 151 with a connector 153 that mating connects with a socket (not shown) on the wearable device 102 such that the wearable device 102 has adequate power to, for example, energize GPS functionality on the watch, and or other electrical watch subsystems, e.g. communications capability, and cloud functioning capability. The system 1 also provides a failsafe lock and key system and functionality to prevent accidental mis-use of the system by a user. Window 130 is provided so that a user may determine if the cartridge is filled with a drug or medicine or not. In another variant, wearable device 102 is a bracelet without wristwatch functionality.

In broad terms, the system has a housing having a fixation means to a user and an orientation element; the cartridge formed so as to be held in a given orientation by the orientation element with respect to the housing: and control means to activate the cartridge to eject a fluid.

When an ejection takes place, the ejection is optionally reported via wireless connection to a network such as the cloud.

The cartridge matingly engages with the housing, optionally via a keyed cartridge surface to a key hole cartridge receiver recess, the keying, of any desired cross-section or form, corresponds to a suitable cartridge type to avoid inadvertent insertion of an improper cartridge, and a retaining device holds the cartridge in place in the housing.

Where the fluid is sensitive to temperature, it is advantageous that the cartridge is receivable into the housing in an insulated recess. A temperature sensor in the housing senses the cartridge and therefore the fluid temperature, optionally indicating the temperature via an indication on the housing. When the temperature sensed is outside an acceptable range for an unacceptable amount of time, a locking device in the system prevents the ejecting of the fluid and an indicator indicates a warning that fluid should not be injected. Preferably, an override device allows a user to override the warning.

The sensed temperature is transmitted to a controller that controls heat flow to or heat removal from the cartridge in order to maintain the fluid temperature within a defined range. The insulated recess may then optionally be temperature controlled to preserve the fluid.

Upon actuation, the fluid in the cartridge is ejected from the cartridge and, where the device is an injector, injected transdermally in the user. On variant of the system is adapted for emergency rescue fluid(s) transdermal delivery, wherein the cartridge is a removable, single use emergency rescue fluid(s) dispensing cartridge, and wherein the system further comprises a wearable device into which the emergency rescue fluid(s) dispensing cartridge uniquely, matingly and removably inserts.

The system further optionally comprising a communication pathway between the wearable device and at least one other network node such as the cloud or other wireless of fixed network.

Preferably, at least one communication pathway exists between the wearable device and at least one GPS network node.

In an aspect of the invention, a control system includes a controller, sensors, and actuation means. The controller is encoded with instructions to identify and manage the fluid, including managing, according to a stored treatment plan devised for the user, the frequency and timing of injections, temperature and storage life of the fluid, composition of the fluid, and/or allergetic conditions of the patient, as well as storing injection history. The invention, comprising a programmable memory and an input means (which is preferably remote via a Bluetooth connection to a smartphone having a touch screen, via a touch screen of an Apple iWatch, a laptop keyboard or remotely by a medical professional via a network connection, even a connection via the Cloud) allows a user or medical professional to program the device with a user profile, including allergies to specific medicines or antibiotics. Where the system reads a user profile that indicates the user is known to have an allergy to the fluid, then the fluid is not injected without a warning allowing manual override by the user. A display (not shown) such as a small rectangular display (for example, twice the size of the indicated marked “Cloud” in FIG. 1, on the right side rather then the left side of the watch face) integrated in the face of a wristwatch, or other sensory announcement system such as a recording, optionally provides recommendations for the user suggesting when to manually actuate the device. je pense aussi à un profit de l'utilisateur qu'on peut introduire dans le système de deux manières: 1. Comme on le fait avec des montres de sport, en pressant sur des boutons sur la montre, ou en utilisant un écran de type apple watch, ou en communiquant ces données depuis son smartphone)

The control system recognizes the cartridge via RFID or by other means, such as electronic means, such a reading a mechanical key form or stored electronic identifier, and manages the system consistent with the cartridge identified.

A sensor, adapted to read a characteristic of the user, communicates sensed information to the control system. The sensor may be a gas sensor (such as a nerve gas sensor), or a pulse or body temperature sensor.

The housing preferably includes a GPS transmitter communicatively connected to the network to transmit a location of the user at a determined interval or time, or triggered by an event.

Components of the control system may optionally be moved out of the housing and into a separate device, to minimize the complication of the portion of the system worn by the user. Preferably, components of the control system reside in a network, such as the cloud, which is connected via a wireless channel to the system. The components of the control system may reside in a smartphone or external computer, by which GPS signals may be communicated by the smartphone or external computer under the control of the controller. This enables the user worn device to be lighter and require less battery life or storage capacity. The smartphone may be connected to the system by a direct connection, such as via a cable, or wireless connection, such as Bluetooth. The control system is programmable to manage the invention to deal with allergetic and other medical conditions of the user, administering the fluid or drug in appropriate doses, at appropriate intervals and times, drugs or medications appropriate to the user to treat the user. The transmission preferably conforms to any of the the medical device connectivity norms defining such transmissions, such as CEN ISO/IEEE 11073, IEE 802.11, DEC, ACM, DEC-SPD, PIV, RTM and IDCO. See Witonsky, P. (2012). Leveraging EHR investments through medical device connectivity. Healthcare Financial Management 66 (8): 50-3, the content of which is incorporated herein by reference thereto.

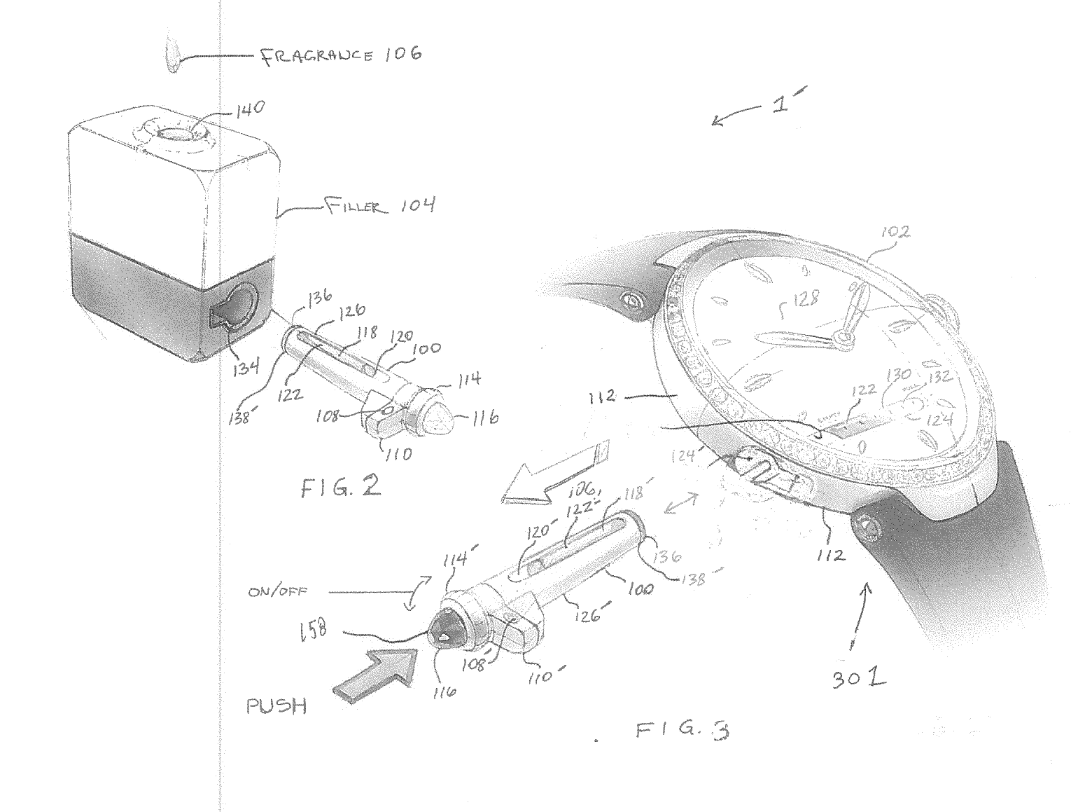

Referring now to FIG. 2-3, in another variant, the system 1′ of the invention is used for fragrance atomization. This embodiment includes: a removable fragrance dispensing cartridge 100′, a wearable device into which the fragrance dispensing cartridge matingly and removably inserts, and a filler assembly for dispensing into the fragrance cartridge a fragrance. Spare cartridges may be stored in storage recesses on a holding strap attached to the housing. Preferably, the cartridge has a transparent portion indicating a fill level of the fluid in the cartridge and wherein at least a portion of the transparent portion is visible through the housing.

The view of the cartridge 100′, an end of the cartridge, on which the fluid is ejected, may be repositioned by turning a swivel head to change the direction of the ejection, from against the skin to away from the skin, 180 degrees perpendicular thereto, or any position in between.

System 1′ provides a removable fragrance cartridge 100′, a wearable device 102 into which the fragrance dispenser 100′ matingly and removably inserts, and a filler assembly 104 for dispensing into the fragrance dispenser 100′ a fragrance 106.

The cartridge 100′ also include an aerosolizator 108′. The aerosolizator 108′ is selected from the group consisting of a nozzle 152, a plurality of orifices, a single orifice, and a porous membrane. In other variants, a vaporizer or other assembly is used to release a fragrance 106 into the atmosphere around a user. Cartridge 100′ includes a protrusion 110′ which extends outside of the housing 112′ of the wearable device. The protrusion 110′ further includes an actuator 114′. The actuator 114′is a rotatable actuator, a fragrance dispensing manual actuator, a manually actuated pump, or in another variant, the actuator 114′ is an on/off actuator. The actuator 114′ and or the cartridge 100′ further includes an ornamental crown 116′. The ornamental crown 116′ is selected from the group consisting of a non-precious metal, a precious metal, a natural jem stone, a synthetic jem stone, a diamond. The cartridge 100′ further includes a transparent viewing window 118′ and includes an interior chamber 120′. The interior chamber 120′ further includes a fluid 122′. The fluid 12′ is selected from the group consisting of a fragrance, a concentrated fragrance, a woman's fragrance, a men's fragrance, a pheromone containing fluid, and/or a volatile fragrance.

The cartridge 100′ is configured so that functionality of the cartridge 100′ for vaporizing fluid 122′ is completely independent of the functioning of the wearable device 102′.

As shown in FIG. 3, the wearable device 102′ further includes a chamber 124′ which matingly and removably couples with an exterior housing 126′ of cartridge 100′. The chamber 124′ further includes a fluid tight chamber assembly within an interior housing portion of the wearable device 102′. The exterior housing 126 of cartridge 100 is sealed and fluid tight. The wearable device 102′ further includes a watch dial surface 128 further comprising a transparent portion 130, e.g. also including wording such as full or empty. The transparent portion 130 further includes a plurality of markings 132 indicating the amount of fluid 122 remaining in cartridge 100. The chamber 124 further includes a biased spring and piston assembly (shown in FIG. 1) for exerting force upon the fluid 122′ to assist in the expulsion of the fluid 122′ from aerosolizator 108′.

The filler 104 further includes an interior chamber assembly 134 sized and dimensioned to matingly and removably couple with cartridge 100′ exterior housing 126′ and protrusion 110′. The interior chamber assembly 134 further includes one or more needles (not shown) which are used to fill the cartridge 100 with fluid 122 through a septum 136 on distal end 138′ of cartridge 100′. The filler 104 further includes a manual filling assembly for dispensing fluid 122′ into cartridge 100′ upon insertion of cartridge 100′ into the filler 104. The filler 104 further includes an automated filling assembly for dispensing fluid 122′ into cartridge 100′ upon insertion of cartridge 100′ in the filler 104. In one embodiment, the fluid tight chamber assembly further includes a bellows and piston assembly, comprising a piston and at least one bellows. In a variant, the bellows is a reservoir for the fluid 122′. In another variant, the filler 104 further includes an entrance orifice 140 for entry and temporary storage of fluid 122′. The cartridge 100′ further includes a manual push actuator 144 for actuating external vaporization of fluid 122′. In yet a another variant, the cartridge 100′ further includes a piston and spring assembly 146 comprising a piston 148 for pressurizing fluid 122′ within the internal chamber of the cartridge 100′.

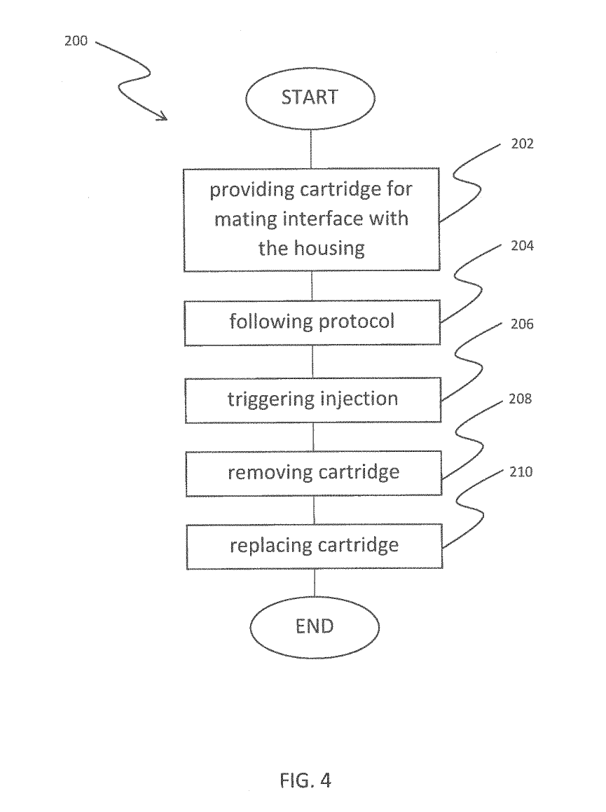

Referring now to FIG. 4, a method 200 of operating at least a portion of an emergency rescue fluid(s) dispensing timepiece system 1 is also provided herein. In a first step 202, the method provides a micro-needle delivery and an emergency rescue fluid(s) dispensing cartridge 100 which matingly and removably fits into the sealed fluid tight chamber 124 by way of a lock and key configuration of the chamber 124 and the cartridge 100. In a second step 204, a prescribed protocol is executed and the user monitored. In a third step 206, a trigger event detected by a controller triggers an injection via a suitable actuator connected thereto and integrated into the cartridge 100 or the wearable device 102. In a fourth step 208, the cartridge 100 is ejected and disposed of. In a fifth step 210, a new cartridge 100 is inserted and the system is ready for operation once again.

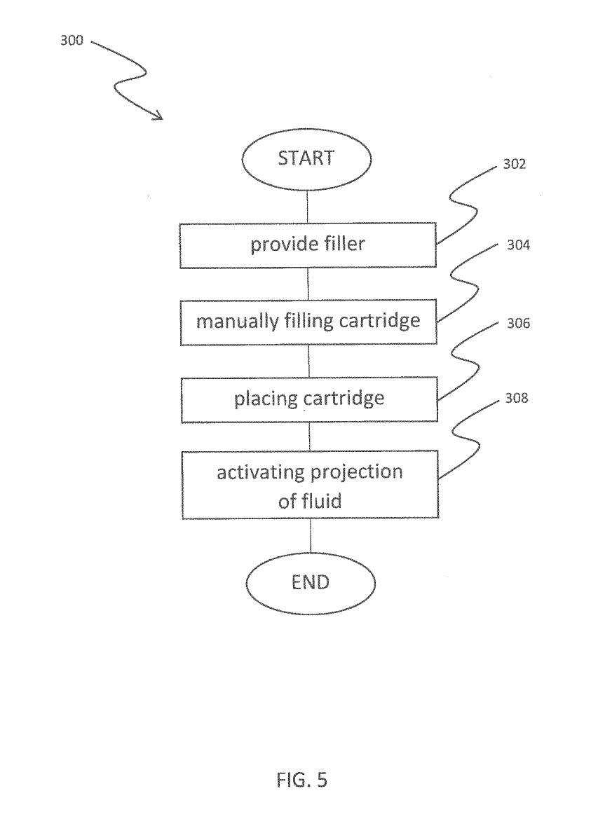

Referring now to FIG. 5, a method 300 of operating at least a portion of a fragrance dispensing timepiece system 301 is also provided herein. The method 300 includes two basic steps. In a first step 302, a wearable device 102′ is provided with a sealed fluid tight chamber 124′. In a second step 304, a fragrance dispensing cartridge 100′ is provided which matingly and removably fits into the sealed fluid tight chamber 124′ of the wearable device 102′ wherein the cartridge 100′ operates completely separately from the typical functions of a wristwatch or an element thereof.

The method 300 further includes one or more of these steps, alone or in combination. In a first step 302, a filler 104 of the cartridge 100′ is provided for filling the cartridge 100′ with a fluid 106, manually filling the cartridge 100′ with fragrance 106 via filler 104 by manually pressing cartridge 100′ into the filler 104′. In a second step 304, the cartridge 100′ is manually filled further by inserting a needle into a septum 136 of the cartridge 100′ thereby filling a reservoir of the cartridge 100′ by pushing a button 158 on the cartridge 100′ such that the manual force on the button 158 such that the manual force on the button fills the cartridge 100′ with the fluid 122′ from the filler 104. In a third step 306, the user places the cartridge 100′ in the chamber 124′. In a fourth step 308, the cartridge 100′ is activated to diffuse the fluid 122′ from the cartridge 100′ to the exterior of a user by pushing the button 158 on the cartridge 100′.

Further, the invention should be considered as comprising all possible combinations of every feature described in the instant specification, appended claims, and/or drawing figures which may be considered new, inventive and industrially applicable.

It should be appreciated that the particular implementations shown and herein described are representative of the invention and its best mode and are not intended to limit the scope of the present invention in any way.

As will be appreciated by skilled artisans, the present invention may be embodied as a system, a device, or a method.

The present invention is described herein with reference to block diagrams, devices, components, and modules, according to various aspects of the invention. It will be understood that each functional block of the blocks diagrams, and combinations of functional blocks in the block diagrams, can be implemented by computer program instructions which may be loaded onto a general purpose computer, special purpose computer, or other programmable data processing apparatus to produce a machine, such that the instructions which execute on the computer or other programmable data processing apparatus create enable the functionality specified in the block diagrams.

Moreover, the system contemplates the use, sate and/or distribution of any goods, services or information having similar functionality described herein.

The specification and figures should be considered in an illustrative manner, rather than a restrictive one and all modifications described herein are intended to be included within the scope of the invention claimed. Accordingly, the scope of the invention should be determined by the appended claims (as they currently exist or as later amended or added, and their legal equivalents) rather than by merely the examples described above. Steps recited in any method or process claims, unless otherwise expressly stated, may be executed in any order and are not limited to the specific order presented in any claim. Further, the elements and/or components recited in apparatus claims may be assembled or otherwise functionally configured in a variety of permutations to produce substantially the same result as the present invention. Consequently, the invention should not be interpreted as being limited to the specific configuration recited in the claims.

In an advantage of the first variant, the invention 102 provides an on-body emergency injection of a fluid that can save the user's life or prevent the consequences of an onslaught of a gas.

In another advantage, the invention 102 is controllable to inject en mass an injection even when the user is incapacitated, in that the medicine or antidote can be administered without user interaction, from a remote station not affected by the gas.

In an advantage of the second variant of the invention 102′, the dispersement of a fragrance can be readily made at a location convenient to a target area of application.

In another advantage, the dispersion of a fragrance can be made at any designated time or at the desire of a third person remote from the wearer, as a forget me not, for example.

In another advantage of the variants of the inventions, they combine functionality with other device to create a solution to a broad range of problems.

Benefits, other advantages and solutions mentioned herein are not to be construed as critical, required or essential features or components of any or all the claims.

As used herein, the terms “includes”, “comprising”, or variations thereof, are intended to refer to a non-exclusive listing of elements, such that any apparatus, process, method, article, or composition of the invention that includes a list of elements, that does not include only those elements recited, but may also include other elements described in the instant specification. Unless otherwise explicitly stated, the use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or adapted by the skilled artisan to other designs without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising ail possible combinations of every feature described in the instant specification, appended claims, and/or drawing figures which may be considered new, inventive and industrially applicable.

Copyright may be owned by the Applicant(s) or their assignee and, with respect to express Licensees to third parties of the rights defined in one or more claims herein, no implied license is granted herein to use the invention as defined in the remaining claims. Further, vis-à-vis the public or third parties, no express or implied license is granted to prepare derivative works based on this patent specification, inclusive of the appendix hereto and any computer program comprised therein.

Additional features and functionality of the invention are described in the claims appended hereto. Such claims are hereby incorporated in their entirety by reference thereto in this specification and should be considered as part of the application as filed.

Multiple variations and modifications are possible in the embodiments of the invention described here. Although certain illustrative embodiments of the invention have been shown and described here, a wide range of changes, modifications, and substitutions is contemplated in the foregoing disclosure. While the above description contains many specific details, these should not be construed as limitations on the scope of the invention, but rather exemplify one or another preferred embodiment thereof. In some instances, some features of the present invention may be employed without a corresponding use of the other features. Accordingly, it is appropriate that the foregoing description be construed broadly and understood as being illustrative only, the spirit and scope of the invention being limited only by the claims which ultimately issue in this application.

What is claimed is:

1. A system for dispensing a fluid comprising
a. a housing having a fixation means to a user and an orientation element;
b. the cartridge formed so as to be held in a given orientation by the orientation element with respect to the housing; and
c. control means to activate the cartridge to eject a fluid.

2. The system of claim 1, when an ejection takes place, the ejection is reported via wireless connection to a network such as a cloud.

3. The system of claim 1, wherein the cartridge matingly engages with the housing, optionally via a keyed cartridge surface to a key hole cartridge receiver recess, the keying, of any desired cross-section or form, corresponding to a cartridge type to avoid inadvertent insertion of an improper cartridge, wherein a retaining device holds the cartridge in place in the housing.

4. The system of claim 1, wherein the fluid is a perfume or pheromone.

5. The system of claim 3, wherein the cartridge is receivable into the housing in an insulated recess.

6. The system of claim 5, wherein a temperature sensor in the housing senses the cartridge and therefore the fluid temperature, optionally indicating the temperature via an indication on the housing.

7. The system of claim 6, wherein when the temperature sensed is outside an acceptable range for an unacceptable amount of time, a locking device in the system prevents the injecting of the fluid and an indicator indicates a warning that fluid should not be injected.

8. The system of claim 7, wherein an override device allows a user to override the warning.

9. The system of claim 6, wherein the sensed temperature is transmitted to a controller that controls heat flow to or heat removal from the cartridge in order to maintain the fluid temperature within a defined range.

10. The system of claim 5, wherein the insulated recess is temperature controlled to preserve the fluid.

11. The system of claim 1, wherein the fluid is ejected from the cartridge and injected transdermally in the user.

12. The system of claim 11, wherein the system is adapted for emergency rescue fluid(s) transdermal delivery, wherein the cartridge is a removable, single use emergency rescue fluid(s) dispensing cartridge, and wherein the system further comprises a wearable device into which the emergency rescue fluid(s) dispensing cartridge uniquely, matingly and removably inserts.

13. The system of claim 12 further comprising a communication pathway between the wearable device and at least one other cloud network node.

14. The system of claim 12 further comprising at least one communication pathway between the wearable device and at least one GPS network node.

15. A control system for use with the device of claim 1, the control system comprising a controller, at least one sensor, and actuation means, a display or other sensory announcement system such as a recording to provide recommendations for the user for actuation, the controller encoded with instructions to identify and manage the fluid, including managing the frequency and timing of ejections, temperature and storage life of the fluid, composition of the fluid, and/or known allergetic conditions of the user patient.

16. The system of claim 15, wherein the control system recognizes the cartridge via RFID or by other means, such as keyed mechanical or electronic means, and manages the system consistent with the cartridge identified.

17. The system of claim 15, wherein the ejection of the fluid from the cartridge is followed by an injection into the user.

18. The system of claim 15, wherein the at least one sensor, adapted to read a physiological or biological characteristic of the body of the user, communicates sensed information to the control system.

19. The system of claim 18, wherein the sensor is adapted to read a chemical characteristic of the surrounding of the user, like a nerve gas sensor.

20. The system of claim 18, wherein the sensor is for pulse or body temperature.

21. The control system of claim 15, wherein components of the control system are moved out of the housing and into a separate device, to minimize the complication of the portion of the system worn by the user.

22. The control system of claim 15, wherein components of the control system reside in a network, such as the cloud, which is connected via a wireless channel to the system.

23. The control system of claim 15, wherein the components of the control system reside in a smartphone or external computer, and wherein GPS signals and the digital identity of the user are communicated by the smartphone or external computer under the control of the controller.

24. The control system of claim 23, wherein the smartphone is connected to the system by a direct connection, such as via a cable, or wireless connection, such as Bluetooth wherein the transmission optionally conforms with a medical norm selected from the group of medical norms consisting of CEN ISO/IEEE 11073, IEE 802.11, DEC, ACM, DEC-SPD, PTV, RTM and IDCO.

25. The control system of claim 15, further programmable to manage allergetic and other medical conditions of the user, administering in appropriate doses, at appropriate intervals and times, drugs or medications appropriate to the user to treat the user.

26. The system of claim 1, wherein the housing includes a GPS transmitter communicatively connected to the network to transmit a location of the user at a determined interval or time, or triggered by an event such as the ejection of the fluid.

27. A system for fragrance atomization, comprising: a removable fragrance dispensing cartridge, a wearable device into which the fragrance dispensing cartridge matingly and removably inserts, and a filler assembly for dispensing into the fragrance cartridge a fragrance.

28. The system of claim 1, wherein spare cartridges may be stored in storage recesses on a holding strap attached to the housing.

29. The system of claim 1, wherein the cartridge has a transparent portion indicating a fill level of the fluid in the cartridge and wherein at least a portion of the transparent portion is visible through the housing.

30. The system of claim 1, wherein the fluid is selected from a group of fluids consisting of peptides, proteins, hormones including insulin, calcitonin, calcitonin gene regulating protein, atrial natriuretic protein, colony stimulating factor, betaseron, erythropoietin (EPQ), interferons such as .alpha., .beta, or .gamma, interferon, somatropin, somatotropin, somatostatin, insulin-like growth factor (somatomedins), luteinizing hormone releasing hormone (LBRH), tissue plasminogen activator (TPA), growth hormone releasing hormone (GHRH), oxytocin, estradiol, growth hormones, leuprolide acetate, factor VIII, interleukins such as interleukin-2, and analogues or antagonists thereof, such as IL-1ra; analgesics such as fentanyl, sufentanil, butorphanol, buprenorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; anti-emetic agents such as scopolamine, ondansetron, domperidone, metoclopramide, and analogues thereof; cardiovascular agents, anti-hypertensive agents and vasodilators such as diltiazem, clonidine, nifedipine, verapamil, isosorbide-5-monotritate, organic nitrates, agents used in treatment of heart disorders, and analogues thereof; sedatives such as benzodiazepines, phenothiazines, and analogues thereof; chelating agents such as defroxanune, and analogues thereof; anti-diuretic agents such as desmopressin, vasopressin, and analogues thereof; anti-anginal agents such as fluorouracil, bleomycin, and analogues thereof; anti-neoplastics such as fluorouracil, bleomycin, and analogues thereof; prostaglandins and analogues thereof; and chemotherapy agents such as vincristine, and analogues thereof, treatments for attention deficit disorder, methylphenidate, fluvoxamine, bisoprolol, tacrolimus, sacrolimus and cyclosporin, vitamins suspended in a liquid carrier, antivenoms, syrums, medications, antibodies, Actemra (tocilizumab), Adcretris (brentuximab vedotin), Arzerra (ofatumumab), Avastin (bevacizumab), Benlysta (belimumab), Cimzia (certolizumab pegol), Erbitux (cetuximab), Herceptin (trastuzumab), Humira (adalimumab), Ilaris (canakinumab), Lucentis (ranibizumab), Mylotarg (gemtuzumab ozogamicin), Perjeta (pertuzumab), Prolia (denosumab), Remicade (infliximab), Simponi (golimumab), Soliris (eculizumab), Stelara (ustekinumab), Tysabri (natalizumab), Vectibix (panitumumab), Xgeva (denosumab), Xolair (omalizumab), Yervoy (ipilimumab), and Zevalin (ibritumomab tiuxetan).
